(19) United States
(12) Patent Application Publication
(10) Pub. No.: US 2019/0114812 A1
(43) Pub. Date: Apr. 18, 2019

(54) METHOD AND ULTRASOUND IMAGING SYSTEM FOR EMPHASIZING AN ULTRASOUND IMAGE ON A DISPLAY SCREEN

(71) Applicant: General Electric Company, Schenectady, NY (US)

(72) Inventor: Paul Mullen, Waukesha, WI (US)

(21) Appl. No.: 15/785,776

(22) Filed: Oct. 17, 2017

(51) Int. Cl. G06T 11/00; A61B 8/00; G06T 3/40

(52) U.S. Cl. CPC G06T 11/001; A61B 8/4427; G06F 3/04817; G06T 3/40; A61B 8/465

(57) ABSTRACT

An ultrasound imaging system and method of emphasizing an ultrasound image includes displaying both the ultrasound image and non-image elements on a display screen while in a normal mode, where the non-image elements have a visual impact of a first level while in the normal mode. The system and method includes automatically entering an image-emphasis mode after a predetermined period of input device inactivity and automatically reducing the visual impact of the non-image elements while in the image-emphasis mode by at least one of automatically reducing a brightness of the non-image elements, increasing a transparency level of the non-image elements, automatically reducing a size of the non-image elements, and automatically increasing a size of the ultrasound image.

### FIELD OF THE INVENTION

This disclosure relates generally to a method and ultrasound imaging system for reducing a visual impact of non-image elements on a display screen after a predetermined period of input device inactivity.

### BACKGROUND OF THE INVENTION

Modern ultrasound imaging systems need to display many different elements on available display screen space. It is typically necessary to display one or more ultrasound images acquired with the ultrasound imaging system as well as a number of non-image elements, such as user interface icons, mode buttons, clinical findings, status buttons, etc. For ultrasound imaging systems that use a touch-based user interface, it may be necessary to display a large number of user interface icons on the display screen in addition to the ultrasound image. The large number of user interface icons and any other non-image elements may distract a user who is trying to view the one or more ultrasound images on the display screen. Depending upon the configuration of the conventional ultrasound system, it may be necessary to display the one or more ultrasound images on only a subset of the total display screen area to allow for the display of the non-image elements. The smaller display size of the ultrasound image may make it harder for a user to quickly and accurately interpret each displayed ultrasound image. The aforementioned issues may be particularly problematic on portable or hand-carried ultrasound imaging systems, due to both the small display screen size of these systems and the increasing prevalence of touch-based user interfaces.

For these and other reasons, an improved ultrasound imaging system and method for emphasizing an ultrasound image on a display screen are desired.

### BRIEF DESCRIPTION OF THE INVENTION

The above-mentioned shortcomings, disadvantages, and problems are addressed herein which will be understood by reading and understanding the following specification.

In an embodiment, a method of emphasizing an ultrasound image includes displaying both the ultrasound image and non-image elements on a display screen while in a normal mode, where the non-image elements have a visual impact of a first level while in the normal mode. The method includes automatically entering an image-emphasis mode after a predetermined period of input device inactivity and automatically reducing the visual impact of the non-image elements while in the image-emphasis mode by at least one of automatically reducing a brightness of the non-image elements, automatically increasing a transparency level of the non-image elements, automatically reducing a size of the non-image elements, and automatically increasing a size of the ultrasound image.

In an embodiment, an ultrasound imaging system includes a memory, an input device, a display screen, and a processor in electronic communication with the memory, the input device, and the display screen. The processor is configured to operate the display screen in a normal mode, where, in the normal mode, both an ultrasound image and non-image elements are displayed on the display screen, where the non-image elements are retrieved from the memory and have a visual impact of a first level in the normal mode. The processor is configured to automatically enter an image-emphasis mode after a predetermined period of input device inactivity, where, in the image-emphasis mode, the processor reduces the visual impact of the non-image elements displayed on the display screen by at least one of automatically reducing a brightness of the non-image elements, automatically increasing a transparency level of the non-image elements, automatically reducing a size of the non-image elements, and automatically increasing a size of the ultrasound image.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a flow chart according to an embodiment; and

FIG. 3 is a schematic representation of a screenshot according to an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical, and other changes may be made without departing from the scope of the embodiments. The following detailed description is, therefore, not to be taken as limiting the scope of the invention.

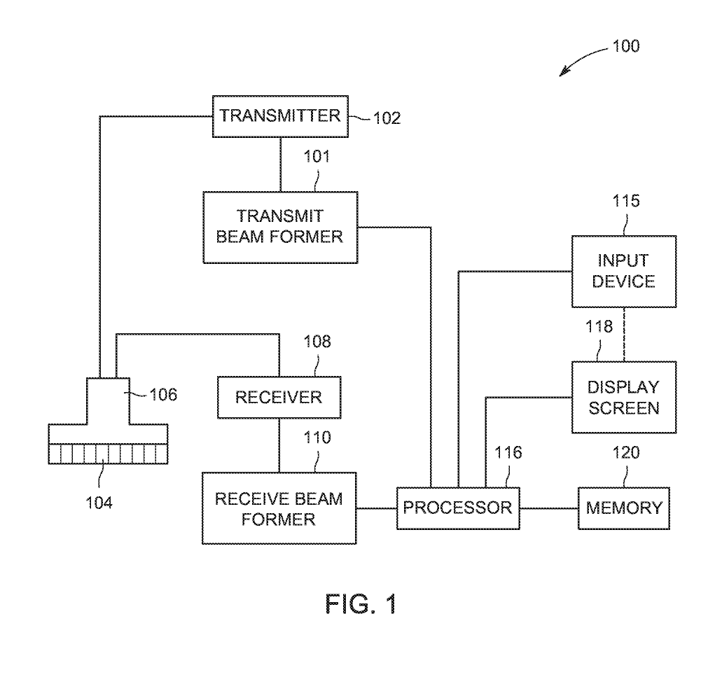

FIG. 1 is a schematic diagram of an ultrasound imaging system 100. The ultrasound imaging system 100 includes a transmit beamformer 101 and a transmitter 102 that drive elements 104 within a probe 106 to emit pulsed ultrasonic signals into a body (not shown). The probe 106 may be any type of ultrasound probe including a linear probe, a sector probe, a convex prove, and a phased array probe. The ultrasound probe 106 may have the elements 104 arranged in a 1D array, a 1.25D array, a 1.5D array, a 1.75D array, or a 2D array. According to an embodiment, the probe 106 may be capable of acquiring real-time 3D ultrasound images. For example, the probe 106 may be a mechanical probe that sweeps or oscillates an array in order to acquire the real-time 3D ultrasound data, or the probe 106 may be a 2D matrix array with full beam-steering in both the azimuth and elevation directions. Still referring to FIG. 1, the pulsed ultrasonic signals are back-scattered from structures in the body, like blood cells or muscular tissue, to produce echoes that return to the elements 104. The echoes are converted into electrical signals, or ultrasound data, by the elements 104, and the electrical signals are received by a receiver 108. The electrical signals representing the received echoes are passed through a receive beamformer 110 that outputs ultrasound data. According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The terms “data” and “ultrasound data” may be used in this disclosure to refer to either one or more datasets acquired with an ultrasound imaging system. The ultrasound imaging system 100 includes an input device 115. The input device 115 may be used to control the input of patient data, or to select various modes, operations, and parameters, and the like. The input device 115 may include one or more of a keyboard, a dedicated hard key, a touch pad, a mouse, a track ball, a rotary control, a slider, and the like. The input device may include a proximity sensor configured to detect objects or gestures that are within several centimeters of the proximity sensor. The proximity sensor may be located on either the display screen 115 or as part of a touch screen. The input device 115 may include a touch screen that is positioned in front of the display screen 118. The combination of the touch screen and the display screen results in a touch-sensitive display screen. The touch screen may, for instance, include a smooth front surface made of glass or plastic and a plurality of touch sensors or proximity sensors located beneath the front surface. Each of the touch sensors may, for instance, be a capacitive sensor or a pressure sensor. Each of the proximity sensors may be an electromagnetic field sensor that works by emitting an electromagnetic field and detecting disturbances in the electromagnetic field caused by the proximity of a user's finger or hand, for instance. According to an embodiment, the processor 116 may be configured to display a plurality of user interface icons on the touch-sensitive display screen that may be activated through interactions with the touch screen. For embodiments where the input device 115 is a touch screen, the user interface may include the combination of the touch-sensitive display screen and user interface icons displayed on the display screen 118. The user interface may also include one or more physical controls (such as buttons, sliders, rotary knobs, keyboards, mice, trackballs, etc.) either alone or in combination with graphical user interface icons displayed on the display screen. According to some embodiments, the user interface may include a combination of physical controls (such as buttons, sliders, rotary knobs, keyboards, mice, trackballs, etc.) and user interface icons displayed on either the display screen 118 or on a touch-sensitive display screen. The display screen 118 may be configured to display a graphical user interface (GUI) from instructions stored in a memory 120. The GUI may include user interface icons to represent commands and instructions. The user interface icons of the GUI are configured so that a user may select commands associated with each specific user interface icon in order to initiate various functions controlled by the GUI. For example, various user interface icons may be used to represent windows, menus, buttons, cursors, scroll bars, etc. According to embodiments where the input device 115 includes a touch screen, the touch screen may be configured interact with the GUI displayed on the display screen 118. The touch screen may be a single-touch touch screen that is configured to detect a single contact point at a time or the touch screen may be a multi-touch touch screen that is configured to detect multiple points of contact at a time. For embodiments where the touch screen is a multi-point touch screen, the touch screen may be configured to detect multi-touch gestures involving contact from two or more of a user's fingers at a time. The touch screen may be a resistive touch screen, a capacitive touch screen, or any other type of touch screen that is configured to receive inputs from a stylus or one or more of a user's fingers. According to other embodiments, the touch screen may be an optical touch screen that uses technology such as infrared light or other frequencies of light to detect one or more points of contact initiated by a user.

According to various embodiments, the input device may include an off-the-shelf consumer electronic device such as a smartphone, a tablet, a laptop, etc. For purposes of this disclosure, the term “off-the-shelf consumer electronic device” is defined to be an electronic device that was designed and developed for general consumer user and one that was not specifically designed for use in a medical environment. According to some embodiments, the consumer electronic device may be physically separate from the rest of the ultrasound imaging system. The consumer electronic device may communicate with the processor 116 thought a wireless protocol, such Wi-Fi, Bluetooth, Wireless Local Area Network (WLAN), near-field communication, etc. According to an embodiment, the consumer electronic device may communicate with the processor 116 through an open Application Programming Interface (API).

The ultrasound imaging system 100 also includes a processor 116 to control the transmit beamformer 101, the transmitter 102, the receiver 108, and the receive beamformer 110. The processor 116 is configured to receive inputs from the input device 115. The receive beamformer 110 may be either a conventional hardware beamformer or a software beamformer according to various embodiments. If the receive beamformer 110 is a software beamformer, it may comprise one or more of the following components: a graphics processing unit (GPU), a microprocessor, a central processing unit (CPU), a digital signal processor (DSP), or any other type of processor capable of performing logical operations. The receive beamformer 110 may be configured to perform conventional beamforming techniques as well as techniques such as retrospective transmit beamforming (RTB).

The processor 116 is in electronic communication with the probe 106. The processor 116 may control the probe 106 to acquire ultrasound data. The processor 116 controls which of the elements 104 are active and the shape of a beam emitted from the probe 106. The processor 116 is also in electronic communication with the display screen 118, and the processor 116 may process the ultrasound data into images for display on the display screen 118. The processor 116 may be configured to display one or more non-image elements on the display screen 118. The instructions for display each of the one or more non-image elements may be stored in a memory 120, which will be described in additional detail hereinafter. For purposes of this disclosure, the term “electronic communication” may be defined to include both wired and wireless connections. The processor 116 may include a central processing unit (CPU) according to an embodiment. According to other embodiments, the processor 116 may include other electronic components capable of carrying out processing functions, such as a digital signal processor, a field-programmable gate array (FPGA), a graphics processing unit (GPU), or any other type of processor. According to other embodiments, the processor 116 may include multiple electronic components capable of carrying out processing functions. For example, the processor 116 may include two or more electronic components selected from a list of electronic components including: a central processing unit (CPU), a digital signal processor (DSP), a field-programmable gate array (FPGA), and a graphics processing unit (GPU). According to another embodiment, the processor 116 may also include a complex demodulator (not shown) that demodulates the RF data and generates raw data. In another embodiment the demodulation may be carried out earlier in the processing chain. The processor 116 may be adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. Real-time volume rates may vary based on the size of the volume from which data is acquired and the specific parameters used during the acquisition. The data may be stored temporarily in a buffer during a scanning session and processed in less than real-time in a live or off-line operation. Some embodiments of the invention may include multiple processors (not shown) to handle the processing tasks. For example, an embodiment may use a first processor to demodulate and decimate the RF signal and a second processor to further process the data prior to displaying an image. It should be appreciated that other embodiments may use a different arrangement of processors. For embodiments where the receive beamformer 110 is a software beamformer, the processing functions attributed to the processor 116 and the software beamformer hereinabove may be performed by a single processor, such as the receive beamformer 110, or the processor 116. Or the processing functions attributed to the processor 116 and the software beamformer may be allocated in a different manner between any number of separate processing components.

According to an embodiment, the ultrasound imaging system 100 may continuously acquire real-time 3D ultrasound data at a volume-rate of, for example, 10 Hz to 30 Hz. A live ultrasound image may be generated based on the real-time 3D ultrasound data. The live ultrasound image may be refreshed at a frame-rate that is similar to the volume-rate. Other embodiments may acquire data and or display the live ultrasound image at different volume-rates and/or frame-rates. For example, some embodiments may acquire real-time 3D ultrasound data at a frame-rate of less than 10 Hz or greater than 30 Hz depending on the size of the volume and the intended application. Other embodiments may use 3D ultrasound data that is not real-time 3D ultrasound data. A memory 120 is included for storing processed frames of acquired data and instructions for displaying one or more non-image elements on the display screen 118. In an exemplary embodiment, the memory 120 is of sufficient capacity to store frames of ultrasound data acquired over a period of time at least several seconds in length. The memory 120 may comprise any known data storage medium. In embodiments where the 3D ultrasound data is not real-time 3D ultrasound data, the 3D ultrasound data may be accessed from the memory 120, or any other memory or storage device. The memory or storage device may be a component of the ultrasound imaging system 100, or the memory or storage device may external to the ultrasound imaging system 100.

Optionally, embodiments of the present invention may be implemented utilizing contrast agents and contrast imaging. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component, and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processor 116 (e.g., B-mode, Color Doppler, M-mode, Color M-mode, spectral Doppler, Elastography, TVI, strain, strain rate and combinations thereof, and the like) to form 2D or 3D images or data. For example, one or more modules may generate B-mode, color Doppler, M-mode, color M-mode, spectral Doppler, Elastography, TVI, strain, strain rate and combinations thereof, and the like. The image beams and/or frames are stored and timing information indicating a time at which the data was acquired in memory may be recorded. The modules may include, for example, a scan conversion module to perform scan conversion operations to convert the image frames from beam space coordinates to display space coordinates. A video processor module may be provided that reads the image frames from a memory and displays the image frames in real time while a procedure is being carried out on a patient. A video processor module may store the image frames in an image memory, from which the images are read and displayed.

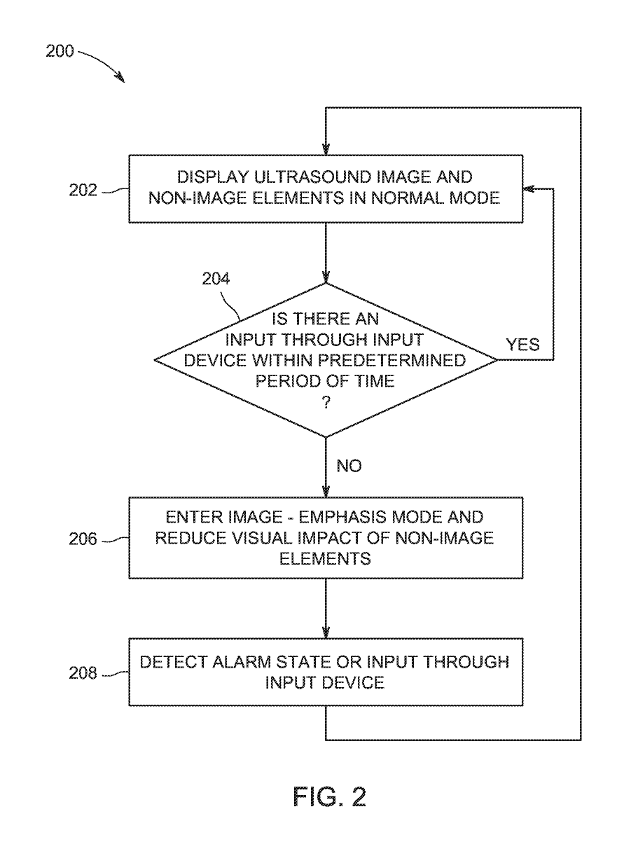

FIG. 2 is a flow chart of a method in accordance with an exemplary embodiment. The individual blocks of the flow chart represent steps that may be performed in accordance with the method 200. Additional embodiments may perform the steps shown in a different sequence, and/or additional embodiments may include additional steps not shown in FIG. 2. The technical effect of the method 200 is the entering of an image-emphasis mode with an ultrasound imaging system after a predetermined period of time without any input through the input device 115.

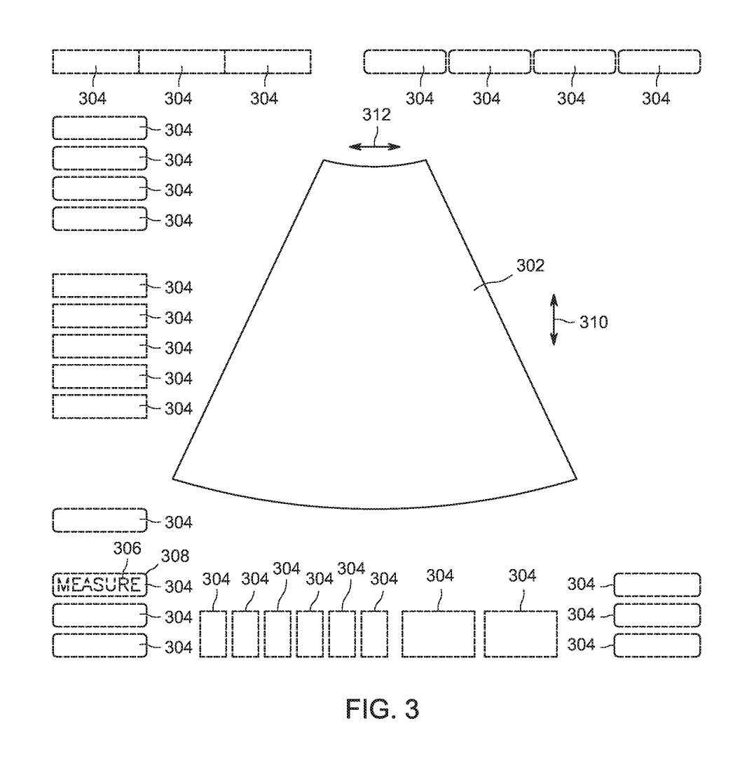

The method 200 will be described with respect to an exemplary embodiment where the method 200 is performed with the ultrasound imaging system 100 shown in FIG. 1. At step 202, the processor 116 displays an ultrasound image 302 (shown in FIG. 3) and a plurality of non-image elements 304 while in a normal mode. The non-image elements 304 are shown in dashed line in FIG. 3, but it should be appreciated that the non-image elements 304 would be displayed in solid line in the normal mode. Non-images elements 304 may include any graphical elements displayed on the display screen that are not an ultrasound image. Some non-limiting examples of non-image elements 304 include user interface icons, status parameters, quantitative results, and labels. Examples of user interface icons include buttons, menus, drop down lists, navigational controls, etc. According to an embodiment, some or all of the non-image elements 304 may include a label, such as exemplary label 306 shown on non-image element 308. According to other embodiments, some or all of the non-image elements 304 may include a graphical symbol representing the function of a particular non-image element. It should be appreciated that some of the non-image elements 304 may include labels while other of the non-image elements may be identified with graphical symbols. Additionally, according to other embodiments, some of the non-image elements 304 may include both a label and a graphical symbol.

The ultrasound image 302 may be accessed from the memory 120 or the ultrasound image 302 may be generated from ultrasound data acquired with the probe 106. The plurality of non-image elements 304 are displayed on the display screen 118 at the same time as the ultrasound image 302 while in the normal mode. The non-image elements 304 have a visual impact of a first level while in the normal mode. The normal mode will be described in additional detail hereinafter.

At step 204, the processor 116 determines if an input has been entered through the input device 115 within a predetermined period of time. The predetermined period of time may be set from the factory or it may be user-configurable. For instance, the predetermined period of time may range from 1 second to several minutes in length. According to an embodiment, the predetermined period of time may be a threshold set to determine when a user is studying the ultrasound image 302 without providing any additional inputs through the input device 115. If there is an input through the input device 115 at step 204, the method 200 returns to step 202 and the ultrasound imaging system 100 remains in the normal mode. However, if there is not an input through the input device 115 within the predetermined period of time, the method 200 advances to step 206.

At step 206, the processor 116 causes the ultrasound imaging system to enter a image-emphasis mode. In the image-emphasis mode, the processor 116 adjusts one or both of the ultrasound image 302 and the non-image elements 304 in order to reduce the visual impact of the non-image elements 304 in the image-emphasis mode. According to an embodiment, the processor 116 reduces the visual impact of the non-image elements 304 from a visual impact of a first level to a visual impact of a second level. During the image emphasis mode, the processor 116 reduces the visual impact of the non-image elements 304 in order to emphasize the ultrasound image 302 on the display screen 118. Many variables may contribute to the overall visual impact of the non-image elements 304. For example, the visual impact of the non-image elements 304 may be determined by one or more variables selected from the following list: a size or area of the non-image elements 304, a brightness of the non-image elements 304, a transparency level of the non-image elements 304, a color of the non-image elements 304, a total size or area occupied by all of the non-image elements 304, and a font size of labels of the non-image elements 304. The visual impact of the non-image elements 304 may be decreased by adjusting one or more of the variables in the following manners: decreasing the size or area of some or all of the non-image elements 304, decreasing the brightness of some or all of the non-image elements 304, increasing a transparency level for some of all of the non-image elements 304, selecting a color that is more subdued for some or all of the non-image elements 304, reducing the total size or area occupied by all of the non-image elements 304, and decreasing the font size of a label of some or all of the non-image elements 304.

According to an embodiment, the processor 116 may reduce the size of some or all of the non-image elements 304 while in the image-emphasis mode. In FIG. 3, all of the non-image elements 304 are shown in dashed-line to indicate that the display qualities of the non-image elements 304 may be adjusted between the normal mode and the image-emphasis mode. While in the image emphasis mode, all the non-image elements 304 (shown in dashed line) may be reduced in size compared to the normal mode. According to an embodiment, where the non-image elements 304 include labels, such as the label 306, a font size of the labels 304 may also be reduced while in the image-emphasis mode. If the total area occupied by the non-image elements 304 on the display screen 118 is reduced, then it is also possible to increase the size of the ultrasound image 302. Accordingly, in some embodiments, the size of the ultrasound image 302 may be increased in the image-emphasis mode. The size of the ultrasound image 302 may be increased in both a depth direction 310 and an azimuth direction 312. The net result of reducing the size of the non-image elements 304 and increasing the size of the ultrasound image 302 is increasing the visual impact of the ultrasound image 302 while decreasing the visual impact of the non-image elements 304. While in the image-emphasis mode, the ultrasound image 302 is more prominent than it is in the normal imaging mode. According to embodiments where the non-image elements 304 are reduced in size in the image-emphasis mode, the non-image elements 304 occupy a much smaller percentage of the available display screen 118. Decreasing the visual impact of the non-image elements 304 makes it easier for the user to focus his or her attention on the ultrasound image 302. In some embodiments, only a subset of the non-image elements 304 may be reduced in size in the image-emphasis mode compared to the normal mode.

According to other embodiments, the processor 116 may reduce the brightness of the non-image elements 304 while in the image-emphasis mode. For example, the non-image elements 304 may be displayed more dimly (i.e., reduced in brightness) compared to the non-image elements 304 in the normal mode. Reducing the brightness of the non-image elements may result in displaying “ghosted” non-image elements 304 that are only faintly discernable from the background of the display screen. Reducing the brightness of the non-image elements 304 emphasizes the ultrasound image 302 and makes it easier for the user to focus on the ultrasound image 302 when interpreting or using the image 302 to make an assessment or diagnosis.

According to an embodiment, a transparency level of the non-image elements 304 may be increased during the image-emphasis mode. The non-image elements 304 may be displayed at a first transparency level during the normal mode and at a second transparency level during the image-emphasis mode during the image-emphasis mode, where the second transparency level is greater than the first transparency level. An alternative way to describe increasing the transparency level would be to say that the opacity of the non-image elements 304 is reduced in the image-emphasis mode compared to the normal mode.

According to another embodiment, the non-image elements 304 may be completely removed (i.e., not displayed) from the display screen during the image-emphasis mode. Additionally, the ultrasound image 302 may be enlarged compared to the normal mode while some or all of the non-image elements are not displayed during the image-emphasis mode. In all of the embodiments, the visual impact of the non-image elements is reduced to a second level that is lower than the first level while in the image-emphasis mode.

It should be appreciated that the various ways described hereinabove of decreasing the visual impact of the non-image elements 304 may be combined with each other. Embodiments may include any combination of increasing the size of the ultrasound image 302, decreasing the size of the non-image elements 304, decreasing the brightness of the non-image elements 304, and decreasing the size of any labels while in the image-emphasis mode.

According to an embodiment, the ultrasound imaging system 100 remains in the image-emphasis mode until either an input is detected by the processor 116 through the input device 115 or an alarm state is detected in the ultrasound imaging system 100. An input may include manipulating a physical control, touching a touch screen or positioning a finger or stylus within a predetermined distance of a proximity sensor. An alarm state may be caused by a safety alarm within the ultrasound imaging system 100 or an alarm from a separate device that is in electronic communication with the ultrasound imaging system, such as a patient monitoring device or other connected device. For example, referring to FIG. 2, either an input or an alarm state is detected at step 208. After detecting the input or an alarm state, the method 200 returns to step 202 and the ultrasound imaging system 100 returns back into the normal mode where the non-image elements are displayed with a visual impact of the first level. According to an embodiment where an alarm state is detected, a non-image element 304 displaying the alarm may be displayed with a visual impact of the first level. According to other embodiments, only a subset of the non-image elements 304 may be displayed with the visual impact of the first level in response to detecting the alarm state. According to an exemplary embodiment, the changes that were applied while transitioning from the normal mode to the image-emphasis mode may be reversed when the method 200 returns to the normal mode. For example, according to an embodiment where the non-image elements 304 were reduced in brightness in the image-emphasis mode, the brightness of the non-image elements 304 may be increased back to the brightness level at which they were originally displayed during the normal mode. Likewise, any changes to the size of the ultrasound image 302, the size of the non-image elements 304, the font size of labels used for the non-image elements, etc. are reversed when the ultrasound imaging system returns to the normal mode. The visual impact of the non-image elements returns to the first level in the normal mode. As described earlier, the input device 115 may be a touch screen and the input may include a touch or gesture of the touch screen. According to other embodiments, the input may be any input provided through a keyboard, a dedicated hard key, a touch pad, a mouse, a track ball, a rotary control, a slider, or a similar physical control.

According to an embodiment, the processor 116 may selectively display one or more non-image elements 304 with a visual impact of the first level to highlight the next step in a workflow. For example, from the image-emphasis mode, where all of the non-image elements 304 displayed with the visual impact of the second level, the processor 116 may adjust the visual impact of one or more non-image elements 304 to indicate the next step or steps to be performed in a workflow. For example, the one or more of the non-image elements indicating the next step or steps may be displayed at a visual impact of the first level. Examples of workflow steps that may be highlighted may include obtaining a measurement, capturing a specific view of the patient, or capturing an image from a particular imaging mode.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they have structural elements that do not differ from the literal language of the claims or if they include equivalent structural elements with insubstantial differences from the literal language of the claims.

We claim:

1. A method of emphasizing an ultrasound image comprising:
displaying both the ultrasound image and non-image elements on a display screen while in a normal mode, where the non-image elements have a visual impact of a first level while in the normal mode;
automatically entering an image-emphasis mode after a predetermined period of input device inactivity; and
automatically reducing the visual impact of the non-image elements while in the image-emphasis mode by at least one of automatically reducing a brightness of the non-image elements, automatically increasing a transparency level of the non-image elements, automatically reducing a size of the non-image elements, and automatically increasing a size of the ultrasound image.

2. The method of claim 1, wherein automatically reducing the visual impact of the non-image elements comprises automatically reducing the brightness of at least one of the non-image elements.

3. The method of claim 2, wherein automatically reducing the visual impact of the non-image elements further comprises automatically increasing the size of the ultrasound image.

4. The method of claim 1, wherein automatically reducing the visual impact of the non-image elements comprises automatically reducing the size of at least one of the non-image elements.

5. The method of claim 4, where at least one of the non-image elements comprises a label and reducing the size of the non-image elements comprises reducing a font size of the label.

6. The method of claim 1, wherein automatically reducing the visual impact of the non-image elements comprises automatically increasing a size of the ultrasound image while not displaying the non-image elements.

7. The method of claim 1, further comprising automatically returning to the normal mode in response to detecting an input through the input device, where the visual impact of the non-image elements is restored to the first level while in the normal mode.

8. The method of claim 7, where at least one of the non-image elements comprises a user interface icon and the input comprises touching a touch screen.

9. The method of claim 8, where the input comprises a gesture performed by an operator within a predetermined distance of the touch screen.

10. The method of claim 7, where the input is performed on a separate device and transmitted through an open API.

11. The method of claim 10, where the separate device comprises an off-the-shelf consumer electronic device.

12. The method of claim 1, where the non-image elements include at least one of a user interface icon, a status parameter, and a label.

13. A ultrasound imaging system comprising:
a memory;
an input device;
a display screen; and
a processor in electronic communication with the memory, the input device, and the display screen;
wherein the processor is configured to operate the display screen in a normal mode, where, in the normal mode, both an ultrasound image and non-image elements are displayed on the display screen, where the non-image elements are retrieved from the memory and have a visual impact of a first level in the normal mode;
wherein, the processor is configured to automatically enter an image-emphasis mode after a predetermined period of input device inactivity, where, in the image-emphasis mode, the processor reduces the visual impact of the non-image elements displayed on the display screen by at least one of automatically reducing a brightness of the non-image elements, automatically increasing a transparency level of the non-image elements, automatically reducing a size of the non-image elements, and automatically increasing a size of the ultrasound image.

14. The ultrasound imaging system of claim 13, where the input device comprises a touch screen positioned in front of the display screen.

15. The ultrasound imaging system of claim 13, wherein the ultrasound imaging system is a handheld ultrasound imaging system.

16. The ultrasound imaging system of claim 13, wherein the processor is configured to automatically reduce the visual impact of the non-image elements by automatically reducing the brightness of the non-image elements.

17. The ultrasound imaging system of claim 16, wherein the processor is configured to automatically reduce the visual impact of the non-image elements by automatically increasing the size of the ultrasound image.

18. The ultrasound imaging system of claim 13, wherein the processor is configured to automatically reduce the visual impact of the non-image elements by automatically reducing the size of the non-image elements.

19. The ultrasound imaging system of claim 13, wherein the processor is configured to return to operating the display screen in the normal mode in response to detecting an input from the input device.

20. The ultrasound imaging system of claim 13, wherein the processor is configured to return to operating the display screen in the normal mode in response to detecting an alarm state.

21. The ultrasound imaging system of claim 20, wherein the alarm state is within the ultrasound imaging system.

22. The ultrasound imaging system of claim 20, wherein the alarm state is generated from a separate device in electronic communication with the ultrasound imaging system.

23. The ultrasound imaging system of claim 13, wherein the processor is configured to increase the visual impact of one or more of the non-image elements to highlight a workflow step from the image-emphasis mode.

24. The ultrasound imaging system of claim 16, wherein the processor is configured to return to operating the display screen in the normal mode in response to detecting an input from the input device, and where the processor is configured to return to operating the display screen in the normal mode by increasing the brightness of the non-image elements.

25. The ultrasound imaging system of claim 17, wherein the processor is configured to return to operating the display screen in the normal mode in response to detecting an input from the input device, and where the processor is configured to return to operating the display screen in the normal mode by increasing the brightness of the non-image elements and decreasing the size of the ultrasound image.
